(19) United States
(12) Patent Application Publication
(10) Pub. No.: US 2009/0081122 A1
(43) Pub. Date: Mar. 26, 2009

(54) Injectable superparamagnetic nanoparticles for treatment by hyperthermia and use for forming an hyperthermic implant

(75) Inventors: Daniel Rufenacht, Geneva (CH); Eric Doelker, Conches (CH); Olivier Jordan, Lausanne (CH); Mathiew Chastellain, Blonay (CH); Alke Petri-Fink, Bussigny (CH); Heinrich Hofmann, Pully (CH)

Correspondence Address: HARNESS, DICKEY & PIERCE, P.L.C., P.O. BOX 828, BLOOMFIELD HILLS, MI, 48303, US

(73) Assignees: UNIVERSITE DE GENEVE, Geneva 4 (CH); ECOLE POLYTECHNIQUE FEDERAL DE LAUSANNE (EPFL), Lausanne (CH); HOPITAUX UNIVERSITAIRES DE GENEVE, Geneva 14 (CH)

(21) Appl. No.: 11/918,927

(22) PCT Filed: May 23, 2005
(86) PCT No.: PCT/EP2005/005553

§ 371 Date: May 23, 2008

(51) Int. Cl. A61K 51/02; A61K 9/00; A61P 35/00; A61P 19/00; A61K 33/26

(52) U.S. Cl. 424/1.29; 424/422; 424/646

(57) ABSTRACT

The injectable formulation for treatment by hyperthermia comprises a liquid carrier and heat-generating superparamagnetic iron oxide nanoparticles having a mean diameter not greater than 20 nm. Said injectable formulation is able to form in-situ a hyperthermic solid or semi-solid implant upon contact with a body fluid or tissue. Said hyperthermic solid or semi-solid implant may be useful for treating a tumor or a degenerative disc disease by hyperthermia.

### FIELD OF THE INVENTION

The present invention concerns an injectable formulation for treatment by hyperthermia, said injectable formulation comprising a liquid carrier and heat-generating nanoparticles, the use of said injectable formulation for forming in-situ an hyperthermic implant upon contact with a body fluid or tissue, said hyperthermic implant and a process for preparing nanoparticles-containing silica beads for use in said injectable formulation.

### BACKGROUND OF THE INVENTION

Proliferative diseases, such as for example, cancer, represent a tremendous burden to the health-care system.

Cancer, which is typically characterized by the uncontrolled division of a population of cells frequently results in the formation of a solid or semi-solid tumor, as well as subsequent metastases to one or more sites.

In addition to surgery, conventional methods of cancer treatment include radiotherapy, which operates to effectuate physical damage to malignant cells so as to render them incapable of cell division, and/or chemotherapy, which generally involves systemically administering cytotoxic chemotherapeutic drugs that alter the normal structure, function or replication of DNA.

However, a problem with these approaches is that radiations in the case of radiotherapy, and chemotherapeutic drugs in the case of chemotherapy, are also toxic to normal tissues, and often create life-threatening side effects.

A very promising therapeutical approach which may be applied either alone or in combination with radiotherapy and/or chemotherapy in the treatment of cancer is hyperthermia, as indicated by recent clinical trials (M. H. Falk, R. D. Issel, “Hyperthermia in oncology”, Int. J. Hyperthermia 17: 1-18 (2001); P. Wust, B. Hildebrandt, G. Sreenivasa, B. Rau, J. Gellermann, H. Riess, R. Felix. P. Schlag, “Hyperthermia in combined treatment of cancer”, The Lancet Oncology, 3: 487-497 (2002); A. Jordan, T. Rheinlander, et al. “Increase of the specific absorption rate (SAR) by magnetic fractionation of magnetic fluids”, Journal of Nanoparticle Research 5 (5-6): 597-600 (2003); A. Jordan, W. Schmidt et al., “A new model of thermal inactivation and its application to clonogenic survival data for human colonic adenocarcinoma cells”, Radiation Research 154(5):600-607 (2000); A. Jordan, R Schlolz, et al., “Pesentation of a new magnetic field therapy system for the treatment of human solid tumors with magnetic fluid hyperthermia”, Journal of Magnetism and Magnetic Materials 225(1-2): 118-126 (2001).

Hyperthermia may be defined as a therapeutical procedure used to increase temperature of organs or tissues affected by cancer between 41 to 46° C. in order to induce apoptosis of cancer cells.

Hyperthermia, when used in combination with radiotherapy, is known to enhance radiation injury of tumor cells, and when used in combination with chemotherapy, is known to enhance chemotherapeutic efficacy.

Further, even mildly elevated temperatures are known to significantly potentiate the effects of radiotherapy and chemotherapy.

Such combinations of treatment modalities could result in lower doses of chemotherapeutic agents or radioactivity necessary to achieve a given effect, thus resulting in less toxicity.

Therefore, using hyperthermia should be considered as an advantageous treatment modality allowing to reduce life-threatening side effects caused by radiotherapy and chemotherapy.

Amongst the various techniques proposed for achieving the required temperature increase, it may be cited for example those reported in details by P. Wust, B. Hildebrandt, G. Sreenivasa, B. Rau, J. Gellermann, H. Riess, R. Felix, P. Schlag, “Hyperthermia in combined treatment of cancer” in The Lancet Oncology, 3: 487-497 (2002) and by P. Moroz, S. K. Jones and Bruce N. Gray, “Status of Hyperthermia in the Treatment of Advanced Liver Cancer”, in J. Surg. Oncol. 77: 259-269 (2001).

However, these various techniques used so far to induce hyperthermia still suffer from significant limitations, the most important of which being a poor control of the heat delivered to the tumor, a poor control of the intratumoral space filling, and a poor control of the precise localization of the hyperthermic effect.

Therefore, providing a hyperthermia technique to reach a controlled temperature at moderate temperatures in a defined tumor target site is a technical challenge still under development.

Some methods for inducing a localized and targeted hyperthermia by using heat-generating nanoparticles have been proposed.

WO-A-01 58458 proposes a method for inducing a localized and targeted hyperthermia in a cell or tissue by delivering nanoparticles of the nanoshell type having a discrete dielectric or semiconducting core section of silica doped with rare earth emitter, or gold sulfide, surrounded by a metal conducting shell layer of gold, to said cell or tissue and exposing said nanoparticles to electromagnetic radiation under conditions wherein said nanoparticles emit heat upon exposure to said electromagnetic radiation. The core and the shell constituting the nanoparticle may be linked by using biodegradable materials such as a polyhydroxy acid polymer which degrades hydrolytically in the body, in order to facilitate the removal of the particles after a period of time.

WO-A-03 055469 discloses a method for inducing a localized and targeted hyperthermia by incorporating into tumor cells, through ionic targeting, nanoparticles of the shell type, having a superparamagnetic core containing iron oxide and at least two shells surrounding said core, more particularly a cationic inner shell and an anionic outer shell, and exposing said nanoparticles to electromagnetic radiation under conditions wherein said nanoparticles emit heat upon exposure to said electromagnetic radiation.

U.S. Pat. No. 6,514,481 proposes the so-called “nanoclinics” that consist in iron oxide nanoparticles in a silica shell and surrounded by a targeting agent, and optionally containing a tracking dye. Application of a constant magnetic field is thought to destroy targeted cells through a magnetically induced lysis—in contrast to the heat generation obtained under an alternative magnetic field.

U.S. Pat. No. 6,541,039 by A. Jordan and coworkers also proposes iron oxide particles, embedded in at least two shells. The outer shell having neutral and/or anionic groups allows an appropriate distribution into the tumoral tissue. The inner shell displays cationic groups to promote adsorption/absorption by the cells. The nanoparticles are injected as a suspension (“magnetic fluid”) and subsequently exposed to an alternative magnetic field for hyperthermic treatment.

However, these methods do not allow to reach a controlled temperature at moderate temperatures in a defined target volume and to repeat the heating procedure in the defined target volume without repeated administration of the formulation containing nanoparticles.

JP-A-10-328314 discloses a shaped material implant which has to be invasively implanted in a bone for being used in hyperthermia treatment, said shaped material implant comprising an alumina powder, a ferromagnetic powder generating heat in an alternating magnetic field comprised of Fe₃O₄ having a diameter over 50 nm, and a polymerized methacrylate monomer.

During their research to overcome the disadvantages of the known hyperthermia techniques, the present inventors have surprisingly found that by providing a specifically designed injectable formulation comprising a polymer-based solution including suspended heat-generating nanoparticles, and by injecting said formulation directly in preexisting tissue spaces of a tumor or heat-sensitive lesion, an in-situ casting of the lesion core may be obtained, and that said implant based on a polymer matrix containing nanoparticles is able to be heated, repeatedly, upon exposure to an external magnetic field.

On the basis of these results, the present inventors have developed a novel hyperthermic implant, formed by injection through direct puncture at tumoral or heat-sensitive site, of a new liquid formulation for minimally invasive image guided treatment of tumoral or heat-sensitive lesions, which allows a confinement of the cytotoxic effects at and near the tumoral or heat-sensitive site, and which increases the efficiency and the safety of the treatment when compared to conventional embolization or hyperthermic procedure.

In contrast to more conventional hyperthermic treatment techniques using invasive probes that may result in local overheating inducing thermoablation and subsequent tissue necrosis, the hyperthermic implant developed by the present inventors delivers a mild heating with typical temperature increase in the range of 5° C. to 10° C.

The new proposed hyperthermic implant also differs from the so-called “magnetic fluids” since the particles are guided by an injectable polymeric matrix that insures a precise localization of all the particles at the tumor site.

### SUMMARY OF THE INVENTION

According to a first aspect, the present invention provides an injectable formulation for treatment by hyperthermia comprising a liquid carrier and heat-generating superparamagnetic iron oxide nanoparticles having a mean diameter not greater than 20 nm, said injectable formulation being able to form in-situ an hyperthermic solid or semi-solid implant upon contact with a body fluid or tissue.

In a preferred embodiment, the heat-generating superparamagnetic iron oxide nanoparticles may have a mean diameter ranging from 5 to 15 nm.

In a further preferred embodiment, the heat-generating superparamagnetic iron oxide nanoparticles may have a span of 1 or less, said span being defined as span=d90%−d10%/d50%, wherein d90%. d10% and d50% are the nanoparticle sizes in diameters, and the given percentage value is the percentage of particles smaller than that size.

The heat-generating superparamagnetic iron oxide nanoparticles are preferably maghemite nanoparticles, magnetite nanoparticles or a mixture thereof.

The heat-generating superparamagnetic iron oxide nanoparticles have preferably a non-spherical shape, wherein the diameter ratio of the larger diameter to the smaller diameter ranges preferably from 1 to 3.

The heat-generating superparamagnetic iron oxide nanoparticles may be coated with a biocompatible polymer.

Alternatively, the heat-generating superparamagnetic iron oxide nanoparticles may be immobilized in organic or inorganic beads.

In a particularly preferred embodiment, the heat-generating superparamagnetic iron oxide nanoparticles may be immobilized in silica beads which preferably have a mean diameter ranging from 20 nm to 1 μm, more preferably from 300 nm to 800 nm.

Silica beads containing iron oxide nanoparticles may be further coated with a biocompatible polymer.

The liquid carrier is preferably based on anyone of a precipitating polymer solution in water-miscible solvent, an in-situ polymerizing or crosslinking compound, a thermosetting compound and an hydrogel, and more preferably based on a precipitating polymer solution in water-miscible solvent consisting in a solution of a preformed polymer in an organic solvent which is able to precipitate in the tissue following exchange of the solvent with surrounding physiological water, thus being able to produce a polymer cast filling the tissue.

The injectable formulation may comprise a radiopacifier, or alternatively the liquid carrier may be based on a radiopaque polymer.

The injectable formulation may further comprise drugs or biopharmaceuticals.

According to a second aspect, the present invention provides a use of the injectable formulation according to the first aspect for forming in-situ an hyperthermic solid or semi-solid implant, preferably an hyperthermic solid or semi-solid implant for treating a tumor or a degenerative disc disease.

According to a third aspect, the present invention provides an hyperthermic solid or semi-solid implant, said implant being formed in-situ upon contact of the injectable formulation according to the first aspect with a body fluid or tissue, when said injectable formulation is injected into a body.

According to a fourth aspect, the present invention provides a process for preparing iron oxide nanoparticles-containing silica beads for use in the injectable formulation according to the first aspect, said process comprising the steps of flocculating iron oxide nanoparticles in the presence of a controlled amount of poly(vinyl alcohol) (PVA) in order to give aggregates of iron oxide nanoparticles; and reacting said aggregates of iron oxide nanoparticles with a silica precursor in order to give iron oxide nanoparticles-containing silica beads.

According to a fifth aspect, the present invention provides a method for hyperthermic treatment of a tumor which comprises administering an injectable formulation according to the first aspect at the tumoral site of a mammal body, allowing the liquid carrier of the injectable formulation to operate a phase transformation to form in-situ an hyperthermic implant, and applying an external magnetic field to induce an increase of the temperature of the implant.

Advantages of the present invention will appear in the following description.

The present invention will be now described in a more detailed manner.

### BRIEF DESCRIPTION OF THE FIGURES

FIG. 1 shows the maximum applied magnetic field strengths in dependence of the frequency for an human body.

FIG. 2 illustrates the different steps in the process for preparing iron oxide nanoparticles-containing silica beads.

FIG. 3 represents a schematic view of (a) percutaneous access to the tumoral site; (b) injection with an appropriate needle and precipitation of the liquid implant resulting in tumor plastification; and (c) additional mild hyperthermic effect produced when the implant is subjected to an external magnetic field.

FIG. 4 represents a diagram showing the radiopacity increasing with nanoparticles contents.

FIG. 5 is a photography of sections of an embolized mouse tumor showing the intratumoral distribution of an hyperthermic implant.

FIG. 6 is a fluoroscopic image of a dog prostate filled with a radiopaque hyperthermic implant.

FIG. 7 represents a diagram showing the release of a model drug (BSA) from an hyperthermic implant.

### DETAILED DESCRIPTION OF THE PRESENT INVENTION

The injectable formulation for treatment by hyperthermia according to the present invention comprises a liquid carrier and heat-generating superparamagnetic iron oxide nanoparticles having a mean diameter not greater than 20 nm, said injectable formulation being able to form in-situ an hyperthermic solid or semi-solid implant upon contact with a body fluid or tissue.

Iron oxide nanoparticles having a mean diameter greater than 20 nm are not appropriate because they do not exhibit a superparamagnetic behaviour with high magnetic saturation and high magnetic anisotropy in the range from 10,000 J/m³ to 50,000 J/m³ and therefore cannot generate mild heating in an alternate magnetic field suitable for human treatment.

The maximal applied magnetic field strength acceptable for human bodies has to choose in that way that the induced eddy current generates a heat production less than 25 W/l.

This is possible if the frequency of the alternate field is controlled.

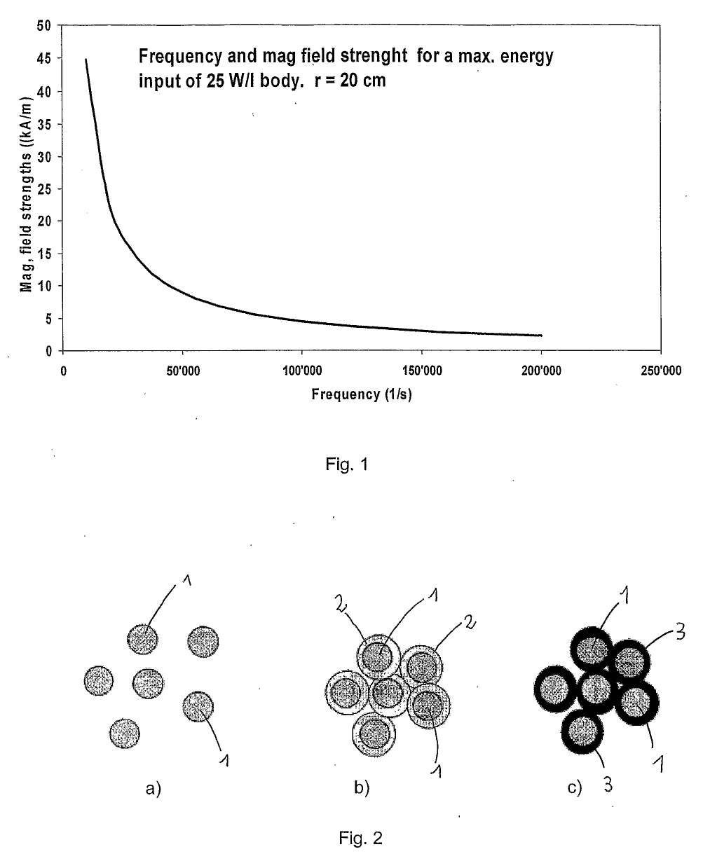

As an example, FIG. 1 shows the maximum applied magnetic field strengths in dependence of the frequency for a human body (diameter 40 cm) and an assumed electrical conductivity of the body of 0.4 S/m, as disclosed by A. Jordan, P. Wurst, R. Scholz, H. Faehling, J. Krause, R. Felix, in “Scientific and Clinical Application of Magnetic carriers” Editors U. Haefeli, W. Schütt, J. Teller, M. Zborowski, Plenum Press, New York, 1997, page 569-595.

For example, for frequency of 50 kHz, a maximal magnetic field strength of 10 kA/m is allowed, higher frequencies lead to a lower field strength.

The iron oxide nanoparticles have preferably a mean diameter ranging from 5 to 15 nm with a narrow size distribution which may be expressed by a span value of 1 or less.

Said span value may be defined as (d10%−d90%)/d50%, d10% representing a size in diameter, wherein 10% of the particles are smaller than this size, d90% representing a size in diameter, wherein 90% of the particles are smaller than this size, and d50% representing a size in diameter, wherein 50% of the particles are smaller than this size.

According to the present invention, a span value of 1 or less warrants an efficient heat generation when a magnetic flux density in the range of 3 to 30 mT (corresponding to 2.388 kA/m to 28 kA/m) with a frequency in the range of 100 to 500 kHz is applied.

The final size will depend on the frequency of the applied alternate magnetic field.

For the purpose of the present invention, said iron oxide nanoparticles are preferably maghemite nanoparticles, magnetite nanoparticles or a mixture thereof.

In a preferred embodiment, said iron oxide nanoparticles may have a non-spherical shape, more preferably with a diameter ratio of the larger diameter to the smaller diameter ranging from 1 to 3 in order to exhibit higher anisotropy constant.

Iron oxide nanoparticles for use in the present invention may be prepared according to a classical wet chemical process for preparing iron oxide nanoparticles, for example a process such as disclosed by A. Bee and R. Massart in Journal of Magnetism and Magnetic Materials, Vol 122, 1, (1990) including steps of alkaline co-precipitation of ferric and ferrous chlorides in aqueous solution, cleaning, thermochemical treatment, and centrifugation.

In one embodiment of the present invention, said iron oxide nanoparticles may be coated with a biocompatible polymer to improve their biocompatibility.

Said coated iron oxide nanoparticles may be obtained by a conventional process of coating with a known bicocompatible polymer.

Alternatively, in another embodiment of the present invention, said iron oxide nanoparticles may be immobilized in inorganic or organic beads to allow a heat generation based on Neel's relaxation, which in turn insures a reproducible heat production.

Organic beads may be based on water-insoluble polymers or on water-soluble polymers.

Said water-insoluble or water-soluble polymers include, for example, vinylic polymers such as poly(vinyl alcohol) or poly(vinyl acetate), cellulose and its derivatives such as cellulose acetate, cellulose acetate butyrate, cellulose acetate propionate, methyl cellulose, hydroxypropyl cellulose, hydroxyethyl cellulose, hydroxypropylmethyl cellulose, or carboxymethyl cellulose; acrylics such as poly(ethyl methacrylate), poly(methyl methacrylate), Eudragit™ or poly(hydroxyl ethyl methacrylate); polyurethanes, polycarbonates, polyethylenes, polyacrylamides, poly(amino acids), biodegradable polymers such as poly (hydroxy acids) or polyorthoesters; and copolymers thereof.

Inorganic beads may be based on silica, calcium phosphates (including hydroxyapatite, tricalcium phosphates), calcium carbonates or sulfates, as well as on biocompatible oxides such as titanium, zirconium or alumina oxides, or mineral glasses (such as Bioglass™).

In a particularly preferred embodiment of the present invention, said iron oxide nanoparticles may be immobilized in silica beads.

Said silica beads immobilizing the iron oxide nanoparticles, also designated herein as “iron oxide nanoparticles-containing silica beads” should have a mean diameter ranging preferably from 20 nm to 1 μm, and more preferably from 300 nm to 800 nm.

Said iron oxide nanoparticles-containing silica beads for use in the present invention may be prepared from iron oxide nanoparticles according to a new process which forms part of the present invention.

Said new process for preparing iron oxide nanoparticles-containing silica beads comprises the steps of:

flocculating iron oxide nanoparticles in the presence of a controlled amount of poly(vinyl alcohol) (PVA) in order to give aggregates of iron oxide nanoparticles,

reacting said aggregates of iron oxide nanoparticles with a silica precursor in order to give iron oxide nanoparticles-containing silica beads.

In a more detailed manner, as illustrated in FIG. 2, the flocculation of iron oxide nanoparticles 1 as illustrated in FIG. 2a) is carried out in a suspension containing a controlled amount of poly (vinyl alcohol) (PVA) to give aggregates of iron oxide nanoparticles, wherein each primary iron oxide nanoparticle 1 is coated with PVA 2, as illustrated in FIG. 2b).

Flocculation of iron oxide nanoparticles is strongly influenced by the presence of PVA in the medium because PVA adsorbs onto the surface of iron oxide nanoparticles and stabilizes them against flocculation.

Controlling the amount of PVA contained in the suspension allows to control the size of the aggregates of primary iron oxide nanoparticles.

Amount of PVA added to the suspension will be chosen from case to case, taking into account that a low content of PVA based on iron oxide will lead to large agglomerates having a size greater than 800 nm and that a high content of PVA based on iron oxide will lead to small agglomerates having a size lower than 50 nm.

However, in a preferred embodiment, weight ratio of PVA to iron oxide should range preferably from 0.01 to 1, and more preferably from 0.1 to 0.43.

PVA used in said new process according to the present invention has a molecular weight ranging preferably from 10 kD to 100 kD, and more preferably from 12 kD to 20 kD and has preferably a degree of hydrolysis ranging from 50% to 100%, more preferably from 83% to 89%.

In a particularly preferred embodiment, the suspension from which iron oxide nanoparticles are flocculated comprises a mixture of water, ethanol, ammonia and PVA.

The water, ethanol and ammonia contents are preferably 25.7, 8.0 and 0.9 M respectively, whereas the ethanol content can be varied from 1 to 16 M and the ammonia content may be varied from 0.1 to 2 M.

Then, the aggregates of iron oxide nanoparticles are reacted with a precursor of silica, for example tetraethoxysilane (TEOS) in order to obtain iron oxide nanoparticles-containing silica beads as illustrated in FIG. 2c) without loosing the structure or size.

In this step, silica forms at the iron oxide nanoparticle surface leading to a highly opened structure made of several silica coated iron oxide nanoparticles linked together by silica “bridges”.

This method advantageously leads to a complete coating of each primary nanoparticle 1 by silica 3, which is important for the magnetic properties since the isolation of each nanoparticle in the aggregate guarantees the superparamagnetic behaviour also in the aggregated form.

The precursor of silica is added at a concentration ranging preferably from 0.01 to 2 M, and more preferably from 0.03 to 0.06 M.

The reaction is carried out preferably under stirring, at a temperature ranging preferably from room temperature to 60° C. for a time ranging preferably from 30 to 300 min.

Iron oxide nanoparticles-containing silica beads will be usually further submitted to conventional cleaning and dialysing steps before their incorporation to the injectable formulation according to the present invention.

In a particular embodiment, said iron oxide nanoparticles-containing silica beads may be further coated with a biocompatible polymer to improve their biocompatibility.

Said coated iron oxide nanoparticles-containing silica beads may be obtained by a conventional process of coating with a known biocompatible polymer.

The liquid carrier of the injectable formulation of the present invention acts as a carrier for the iron oxide nanoparticles or iron oxide nanoparticles-containing silica beads and is able to form in-situ a solid or semi-solid implant retaining iron oxide nanoparticles upon contact with a body fluid or tissue.

Solid or semi-solid implant formed in-situ upon contact with a body fluid or tissue after injection of the injectable formulation of the present invention is able to deliver the heat-generating iron oxide nanoparticles to the targeted site pathological tissues while contributing to the therapeutic effect by plastification of pathological tissues and by retaining the heat-generating iron oxide nanoparticles at the targeted site.

The liquid carrier of the injectable formulation of the present invention which is able to form in-situ a solid or semi-solid implant upon contact with a body fluid or tissue when injected into a body and which incorporates the iron oxide nanoparticles or iron oxide nanoparticles-containing silica beads may be based on

(i) precipitating polymer solutions in water-miscible solvents,

(ii) in-situ polymerizing or crosslinking compounds,

(iii) thermosetting compounds,

(iv) hydrogels.

In a preferred embodiment of the present invention, the liquid carrier of the injectable formulation of the present invention is based on precipitating polymer solutions in water-miscible solvents.

In this preferred embodiment, the liquid carrier consists in a solution of a preformed polymer in an organic solvent that precipitates in the tissue following exchange of the solvent with surrounding physiological water, thus producing a polymer cast filling the tissue.

Such a liquid carrier is designed in the following also as a “precipitating polymer solution”.

Since the precipitation occurs preferentially at the interface between the polymer and the physiological fluids, these precipitating agents tend to reduce the risk of venous leakage when compared to others systems.

The liquid carrier should have a viscosity suitable for injection, that can be controlled either by changing the polymer concentration or by changing the molecular weight of the polymer.

The organic solvents used should preferably have either clinical or pharmaceutical precedents, such as dimethyl sulfoxide (DMSO), ethanol, aqueous solutions of acetic acid, dimethyl isosorbide (DMI), pyrrolidones such as N-methylpyrrolidone (NMP) or 2-pyrrolidone, glycofurol, isopropylidene glycerol (Solketal), ethyl lactate, glycerol, polyethylene glycol, propylene glycol or polyglycols, as well as lipohilic solvents such as triethyl citrate, benzyl alcohol or benzyl benzoate.

Aqueous solutions and mixtures of the above mentioned organic solvents may be used as well.

Preferably, NMP or DMSO is used.

The polymers to be dissolved in the above mentioned solvents include cellulose and its derivatives, such as cellulose acetate, cellulose acetate butyrate, cellulose acetate propionate; acrylics such as poly(methyl methacrylate), poly(ethyl methacrylate), poly(hydroxylethyl methacrylate); polyethylenes, vinylic polymers such as poly(vinyl alcohol) or poly(vinyl acetate); ethylene vinyl alcohol copolymers (EVAL); polyurethanes; polycarbonates; polyacrylonitriles; poly(amino acids) and copolymers thereof.

Biodegradable polymers may be used as well, including poly(hydroxy acids), polyorthoesters, poly(anhydrides) based on sebacic acid or other diacids copolymers.

Polymers such as those disclosed by Dunn et al in U.S. Pat. No. 4,938,763 may also be used.

Preferred polymers have a clinical precedence, such as cellulose acetate disclosed by K. Sugiu, K. Kinugasa, S. Mandai, K. Tokunaga & T. Ohmoto “Direct thrombosis of experimental aneurysms with cellulose acetate polymer (CAP): technical aspects, angiographic follow up, and histological study” in J. Neurosurg 83, 531-538 (1995) and by K. C. Wright, R. J. Greff & R. E. Price “Experimental evaluation of cellulose acetate NF and ethylene-vinyl alcohol copolymer for selective arterial embolization” in J Vasc Interv Radiol 10, 1207-1218 (1999)) or poly(ethylene vinyl alcohol) disclosed by W. Taki et al “A new liquid material for embolization of arteriovenous malformations” in American Journal of Neuroradiology 11, 163-168 (1990); by R. Jahan et al. “Embolization of arteriovenous malformations with Onyx Clinicopathological experience in 23 patients” in Neurosurgery 48, 984-995 (2001) and by A. Komemushi et al. “A new liquid embolic material for liver tumors” in Acta Radiol 43, 186-91 (2002)), or biodegradable poly(hydroxy acids).

The precipitating polymer solution is obtained by dissolving the polymer in the solvent in a concentration ranging from 3% to 60% w/w, and preferably from 5% to 20% w/w.

In another embodiment, the liquid carrier of the injectable formulation of the present invention is based on in-situ polymerizing or crosslinking compounds (II).

Examples of in-situ polymerizing or crosslinking compounds may include monomers, prepolymers and eventually initiators.

For example, such in-situ polymerizing or crosslinking compounds may include cyanoacrylate adhesives and their derivatives (e.g. alkyl cyanoacrylates), acrylic-based polymers such as used for orthopedic cements (e.g. methacrylates and acrylic derivatives), or compounds that crosslink through Michael's addition such as those disclosed in WO-A-03 080144.

In another embodiment, the liquid carrier of the injectable formulation of the present invention is based on thermosetting compounds (iii).

Examples of thermosetting compounds which may be used to deliver and localize the iron oxide nanoparticles, include poloxamers and poloxamines, agarose, n-isopropyl acrylamide (NIPAAM) or chitosan-based thermosetting gels such as those disclosed in U.S. Pat. No. 6,344,488 or disclosed in PCT/EP04/002988 (Pseudo-thermosetting neutralized chitosan composition forming an hydrogel and a process for producing the same).

Injectable polymers based on triblock biodegradable copolymers may also be used to produce hyperthermic implants, such as those disclosed in WO-A-99 21908.

In an other embodiment of the present invention, the iron oxide nanoparticles or nanoparticle-containing beads may be incorporated in hydrogel formulations (iv).

Said hydrogel formulations include compounds that can solidify following ionic concentrations or pH changes (examples are the alginate in presence of divalent cations or the polyvinyl acetate latexes disclosed by Sadato, A. et al. (Experimental study and clinical use of poly(vinyl acetate) emulsion as liquid embolization material) in Neuroradiology 36, 634-641 (1994).).

Said hydrogel compounds also include those used for the embolization of lesions such as disclosed in U.S. Pat. No. 6,113,629 for “Hydrogel for the therapeutic treatment of aneurysms”, 5 Sep. 2000).

The injectable formulation according to the present invention has some radiopacity due to the presence of the iron oxide nanoparticles.

However, additional radiopacity may be required, and said additional radiopacity may be obtained by the addition of a radiopacifier in the injectable formulation as known by those skilled in the art.

To achieve this goal, it may be added a metal, an inorganic salt or an organic compound containing heavy elements such as tantalum, tungsten, barium, bismuth, iodine or zirconium.

More specifically, barium sulfate, bismuth oxide, tantalum powder, tungsten powder or zirconium oxide may be used for this purpose, as well as materials disclosed by F. Mottu, D. A. Rüfenacht and E. Doelker (Radiopaque polymeric materials for medical applications—Current aspects of biomaterials research) in Inv. Radiol 34, 323-335 (1999).

Alternatively, radiopacity may be obtained by using a liquid carrier based on radiopaque polymers such as those disclosed by O. Jordan, J. Hilborn, O. Levrier, P. H. Rolland P. H, D. A. Rüfenacht and E. Doelker (Novel radiopaque polymer for interventional radiology) in the 7th World Biomaterials Congress Proceedings, Sydney, p. 706 (2004); by F. Mottu, D. A. Rüfenacht, A. Laurent & E. Doelker (Iodine-containing cellulose mixed esters as radiopaque polymers for direct embolization of cerebral aneurysms and arteriovenous malformations) in Biomaterials 23, 121-131 (2002); and by C. A. Maurer et al. (Hepatic artery embolisation with a novel radiopaque polymer causes extended liver necrosis in pigs due to occlusion of the concomitant portal vein) in J Hepatol 32, 261-268 (2000).

In order to obtain additional therapeutic effect by using the known synergistic effects between hyperthermia and radiotherapy or chemotherapy, the injectable formulation according to the present invention, may further comprise drugs or biopharmaceuticals.

More specifically, the injectable formulation according to the present invention may further comprise active substances such as drugs or biopharmaceuticals (peptides, proteins, nucleotides, genetic material), preferably anticancerous or anti-infectious substances.

These active substances may be incorporated into the injectable formulation either under the form of free substances, polymer-derivatized substances, or embedded in nano- or microcarriers (nanoparticles, microparticles, liposomes, etc.).

Implants formed from said injectable formulation containing drugs or biopharmaceuticals may therefore be used to release drugs or to deliver biopharmaceuticals with the advantageous effect that the drug release/biopharmaceuticals delivery may be enhanced or triggered by the generation of heat, allowing for a localized, controllable therapeutic effect.

The injectable formulation according to the present invention may be used to form in-situ an hyperthermic solid or semi-solid implant for treating a tumor.

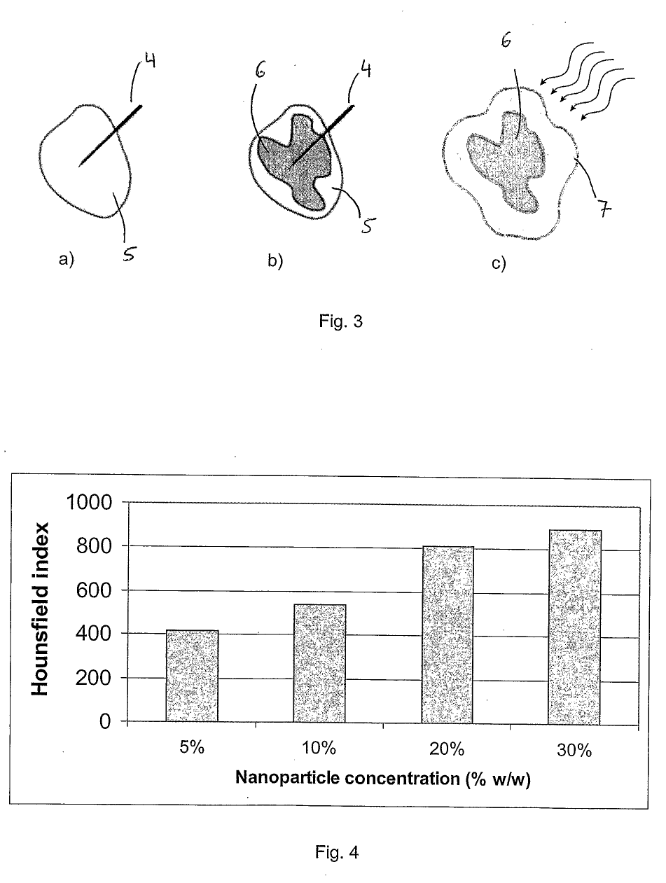

As an example, the injection formulation according to the present invention may be used to form in-situ an hyperthermic solid or semi-solid implant for treating a tumor by a minimally invasive operation according to a procedure which may be illustrated by FIG. 3.

Firstly, a appropriate needle 4 is introduced by direct percutaneous puncture into a tumoral core 5, as illustrated in FIG. 3a).

Secondly, the injectable formulation according to the present invention is injected through the needle 4 to fill the intratumoral space of the tumoral core 5, and then the injectable formulation undergoes a transformation upon contact with the fluid body or tissue to form an hyperthermic solid or semi-solid implant 6, as illustrated in FIG. 3b).

In contrast to conventional endovascular embolization, there is a “plastification” of the lesion and no development of a decaying tumor necrosis.

The implant will carry heat-generating superparamagnetic iron oxide nanoparticles for a mild hyperthermia treatment.

Following formation in-situ of the hyperthermic implant, the remaining tumoral tissue around the implant site can then be heated when the implant is subjected to an alternative magnetic field inducing a mild hyperthermic effect leading to cell death in a rim 7 surrounding the tumor, as illustrated in FIG. 3c).

The heating procedure may be repeated to obtain the desired effect.

Finally, tumoral cell death will result from a combination of intratumoral space filling and localized heating.

In contrast to more conventional hyperthermic treatment techniques using invasive probes that may result in local overheating leading to thermoablation and subsequent necrosis, the hyperthermic implant according to the present invention will deliver a mild heating in view of inducing cell apoptosis.

An originality of the implant according to the present invention is to allow a confinement of the cytotoxic effects at and near the tumoral site, thus increasing the efficiency and the safety of the treatment when compared to conventional embolization or hyperthermic procedures.

Applications may include a variety of tumors since it has been observed that direct puncture procedures may provide access to intra-lesional spaces of many tumors.

Tumor types to which hyperthermic implants of the present invention may be advantageously applied are, for example, rare, highly vascular lesions of the skull base that otherwise need aggressive surgical exposure and carry a high risk of surgical complication, such as seen with glomus tumors; primary and secondary tumor lesion of the spine and pelvis similar to the current acrylic cement implantation (see J. B. Martin, et al., Radiology, 229:593-597 (2003); D. San Millan Ruiz et al., BONE 25:85 S-90S (1999)), but with the potential to offer additional heat treatment; prostate cancer; liver metastases, such as those arising from colorectal cancer.

An hyperthermic solid or semi-solid implant according to the present invention may be used for further applications, for example for treating a degenerative disc disease.

This frequent cause of back pain includes the degeneration of fibrous annular ligaments of the disc allowing for leakage of fragments of disc nucleus leading potentially to nerve root irritation.

Heat treatment is used for disk desiccation and scar induction to avoid further leakage and disc implants may be considered to replace the disc nucleus.

The hyperthermic solid or semi-solid implant according to the present invention may be advantageously used to combine these two treatment forms.

Therefore, in a particular embodiment, the injectable formulation according to the present invention may be used to form in-situ an hyperthermic solid or semi-solid implant for treating a degenerative disc disease, for example disc hernia.

Additional uses of the hyperthermic solid or solid implant according to the present invention may be foreseen for treating any other pathologies which may be treated by hyperthermia.

An additional use of heating material in form of external reusable heat-storing pads as a modality of physical therapy for pain relief may be further foreseen since superficial heat is known to diminish pain and decrease local muscle spasms, such as used in acute low back pain.

The following examples are intended to illustrate the present invention. However, they cannot be considered in any case as limiting the scope of the present invention.

### EXAMPLES

### Example 1

### Iron Oxide Nanoparticles

8.65 g FeCl₃.6H₂O (0.086 M) and 3.18 g FeCl₂.4H₂O (0.043 M) were dissolved in 370 ml ultrapure water under continuous stirring. 30 ml aqueous ammonia (25 vol %) was added in one step while stirring vigorously. A black precipitate formed instantaneously. This precipitate was sedimented on a permanent magnet and the supernatant was removed. The black sediment was washed three times with 400 ml ultrapure water at a time. The final volume of the dispersion was set to 300 ml by adding ultrapure water. The thus obtained dispersion was transferred to plastic centrifugation tubes and was centrifuged at 5000 g for five minutes. The centrifuged solid was placed in a round-bottomed flask. 60 ml of a 0.35 M aqueous Fe(NO₃)₃.9H₂O solution and 40 ml of 2 M nitric acid were added. This mixture was refluxed for 1 hour. During this step the black dispersion turned brown. The mixture was transferred into a beaker which was placed on a permanent magnet and allowed to cool. The supernatant was discarded and 100 ml ultrapure water was added. The thus obtained dispersion was dialyzed against nitric acid (10⁻² M) in suitable dialysis tubes (Sigma Dialysis Tubing, Cellulose membrane, Cut-off >12,000) for 2 days. The nitric acid used for dialysis was changed two times per day. The final product was transferred to plastic centrifugation tubes and was centrifuged at 30,000 g for 15 minutes. The supernatant was collected and will be referred to as “ferrofluid”. The sediment will be referred to as “concentrated ferrofluid”.

Said “ferrofluid” and “concentrated ferrofluid” contained iron oxide nanoparticles exhibiting a mean diameter ranging from 5 to 15 nm with a number weighted average value at 9±1 nm as confirmed by TEM, AFM, XRD and BET. The iron oxide nanoparticles were slightly elongated (ellipsoid) with a diameter ratio of the larger diameter to the smaller diameter of 1.3±0.3. The span was 0.66.

### Example 2

### Iron Oxide Nanoparticles-Containing Beads

### Synthesis Example 1

### a) Polymer Solution:

The polymer solution was prepared by dissolving dry polymer (PVA, Mowiol® 3-83, Clariant) in water and rapidly heating the solution for 15 minutes at 90° C. The polymer concentration of the polymer solution ranged from 0 to 0.2% wt. Ultra-pure water (Seralpur delta UV/UF setting, 0.055 μS/cm) was used in all synthesis steps.

b) 3.3 ml ferrofluid was mixed with 6.6 ml polymer solution in a round-bottomed flask. The mixture was stirred at room temperature for 5 minutes. 10 ml ethanol and 1.5 ml aqueous concentrated ammonia were added while stirring vigorously. The flask was transferred to a thermostat, which was set to 50° C. 250 ml of tetraethoxysilane were injected in this mixture while stirring. The system was stirred for 1 hour at 50° C., then 25 ml ultrapure water was added and the mixture was allowed to cool to room temperature. The size of the so produced iron oxide silica beads was 50 nm.

### Purification

Depending on the initial PVA concentration, the thus obtained dispersion was

a) sedimented on a permanent magnet (low polymer concentration) or

b) centrifuged (high polymer concentration)

For example, an initial polymer concentration of 0.2% wt (Synthesis Example 1) required 30′ centrifugation at 30,000 g. The supernatant was discarded and ultrapure water was added. This procedure was repeated for at least 3 times. The final concentration was adjusted with ultrapure water.

### Synthesis Example 2

1 ml of “concentrated ferrofluid” was dispersed in 20 ml ethanol. 0.1 wt % polymer ((PVA, “Mowiol”, Clariant, 3-83, Mw: 14,000 g/mol, Degree of hydrolysis: 83%) was added. The synthesis was carried out according to the procedure of Synthesis Example 1 as described above. The size of the so produced beads was 200 nm.

### Synthesis Example 3

1 ml of “concentrated ferrofluid” was dispersed in 20 ml ethanol. No polymer was added. The synthesis was carried out according to the procedure of Synthesis Example 1 as described above. The size of the so produced beads was 600 nm.

### Example 3

### Injectable Formulation Containing Iron Oxide Nanoparticles-Containing Beads and Implant

An ethylene-vinyl alcohol copolymer with 44% ethylene contents (EVAL E-105 B, EVAL Europe, Belgium) was dissolved in DMSO (8 g polymer/100 ml DMSO). Iron oxide nanoparticles (NP, diameter <15 nm), embedded in a silica matrix (beads with diameter <1 μm), were suspended in the polymer solution by thorough vortexing and sonication. NP contents of 5% to 30% w/w yielded formulations injectable through a 18G syringe. Precipitation in phosphate buffer, pH 7.2 produced a soft mass adequate for tumor plastification. Following a one-month incubation in the precipitation buffer, no nanoparticle release could be seen by visual inspection. Spectrophotometric measurement of the supernatant indicated that less than 1% of the iron oxide nanoparticles were released (value within measurement error). Therefore, no indication of nanoparticle release was seen in vitro.

### Example 4

### Implant Compatibility with Image Guidance Techniques

The implant of EXAMPLE 3 was examined under computerized tomographic scanner (CT-scan) to measure its radiopacity. It was visible under X-ray imaging, the visibility increasing with NP contents, as illustrated in FIG. 4. In order to improve radiopacity, 10% barium sulfate was added, resulting in highly radiopaque compound (2800 Hounsfield degrees). This latter formulation offered an inhomogeneous radiopacity with a speckled appearance under fluoroscopy, allowing to visualize the flow of the injected liquid into the tissues. Alternatively, polymers grafted with iodinated groups (44% iodine w/w) may be used to improve radiopacity (2300 Hounsfield degrees).

### Example 5

### Injectable Formulation Containing Iron Oxide Nanoparticles Without Silica Beads and Implant

Ferrofluid was freeze dried and the so prepared iron oxide nanoparticles (NP, diameter <15 nm) were suspended in DMSO by thorough vortexing and sonication. An ethylene-vinyl alcohol copolymer with 44% ethylene contents was then dissolved in this suspension (8 g polymer/100 ml DMSO). NP contents of 5% to 30% w/w yielded formulations injectable through a 18G syringe. Precipitation in phosphate buffer, pH 7.2 produced a soft mass adequate for tumor plastification. Following a 1-month incubation in the precipitation buffer, no nanoparticle release could be seen by visual inspection or spectrophotometric measurement. Radiopacity was significantly higher than with the silica beads (960 Hounsfield degrees instead of 540 at 10% w/w concentration).

### Example 6

### Use of Other Types of Polymers

Formulations similar to EXAMPLE 3 have been also obtained with polyurethanes (Tecothane 1075D or Tecogel, Thermedics), acrylics (Paraloid A-12, Rohm; poly(methyl methacrylate), Fluka), cellulose acetate (CA-398-3, Eastman), cellulose acetate butyrate (CA 381-0.5, Eastman), polyvinyl acetate (Mowilith 60, Hoechst), polycarbonate-urethane (Aldrich 41, 831-5). All these solutions in DMSO could, when mixed with 10% w/w of either iron oxide nanoparticles embedded in silica matrix (beads) or iron oxide nanoparticles, form a precipitate and are adequate for injection in biological tissue.

### Example 7

### Use of Alternative Solvents

Solvents presenting a better hemocompatibility than DMSO may be used to formulate injectable implants. Polyurethane polymers (Tecothane and Tecogel), dissolved in N-methylpyrrolidone (Tecothane 5% to 10% w/vol, Tecogel 15% to 20% w/vol) and mixed with 10% of iron oxide nanoparticles embedded in a silica matrix (beads) produced soft, coherent precipitate adequate for tissue plastification. Poly(ethyl methacrylate) dissolved in dimethyl isosorbide (DMI) (8 g polymer/100 ml DMI) or in Glycofurol 75 also produced satisfactory formulations.

### Example 8

### Hydrogel-Like Implant

An injectable, slow-gelling nanoparticles-containing alginate formulation was made as follow. An aqueous solution A of 2% w/w sodium alginate (Fluka, Buchs) and 0.5% w/w tri-sodium phosphate were mixed with a solution B containing 10% w/w of calcium phosphate and 10% w/w of iron oxide nanoparticles embedded in a silica matrix. Injection was carried out with a double syringe or with a double lumen catheter. After mixing, slow gelation took place yielding a soft hydrogel within 10 minutes. No release of the nanoparticles could be observed in vitro. Alternatively, a fast-gelling matrix could be obtained by mixing (A) 2% sodium alginate and (B) a 1% to 8% aqueous solution of calcium chloride added with 10% nanoparticles-containing beads, producing a firm gel within seconds.

### Example 9

### Hyperthermic Bone Cement Implant

An acrylic bone cement containing nanoparticles was made from a commercial Simplex™ cement that consists of an acrylic powder (PMMA) and an acrylic monomer. To obtain a 15% w/w cement, 0.45 g of iron oxide nanoparticles (either embedded in silica matrix (beads), or alone) were mixed with 1.6 g of the acrylic powder and 1 ml of acrylic monomer. The cement could be loaded with up to 23% w/w of silica beads containing nanoparticles, or with up to 15% w/w of nanoparticles. The cements were injectable through 18G needles and hardened similarly to normal cements. No release of the nanoparticles could be observed in vitro.

### Example 10

### Injectable Thermosetting Formulation Containing Iron Oxide Nanoparticles

A chitosan formulation was prepared according to prior art (PCT/EP2004/002988 “Pseudo thermosetting neutralized chitosan composition forming a hydrogel and a process for producing the same”). Briefly, a chitosan of 47% deacetylation degree was dissolved in 3 ml of hydrochloric acid 0.03 N. The solution was cooled down at 4° C. One ml of a mixture of propylene glycol or 1,3-propanediol with water in a ratio 3:7 was added under stirring. The solution was then added with 10% to 20% w/w of nanoparticles embedded in silica beads, and the pH was adjusted to 6.8 by addition of NaOH 0.1 M. Final volume was completed to 5 ml with water. The solution was then injected through a 21G needle into a freshly explanted porcine ureter kept at 37° C. in saline. The formation of a stiff gel was observed within 30 min.

### Example 11

### Bioactive Bone Cement Implant

Bioactive cement based on hydroxyapatite powder, carbonated apatite cement, calcium phosphate cements and glass ceramics powders are under investigation or commercially available (e.g. Norian™). Cement combining a bioactive component and a polymer phase are another promising alternative (e.g. Cortoss™). We selected two commercial cements, Norian™ and Cortoss™ that we loaded with up to 20% w/w iron oxide nanoparticles embedded in silica beads or with 20% w/w iron oxide nanoparticles. The cement could be injected through 18G needle and hardened similarly to non-loaded cements.

### Example 12

### Heat Released from the Nanoparticles-Loaded Implants

Selected implants were submitted to alternative electromagnetic field with a frequency of 140 kHz and a magnetic field strength of 4.77 kA/m. The temperature increase was measured in a differential calorimeter, from which the heat produced and material power loss were calculated (J.-C. Barci et al., in Scientific and Clinical Application of Magnetic Carrier, Plenum Press, 1997). The results are given in Table I below. Comparing the power loss between nanoparticles (NP) and nanoparticles embedded in a silica matrix (beads), it appears that the silica embedding provides a much more efficient heating. Silica-embedded nanoparticles had power loss in the 10 to 37 W/g range, values that have been shown to lead to efficient in vivo hyperthermia. Furthermore, the implant matrix significantly influences the power loss, showing the importance of selecting the appropriate implant matrix for hyperthermia.

TABLE I
Power loss of hyperthermic samples

| | NP | | Power loss |
|---|---|---|---|
| | content | | [W/g of |
| Injectable polymer | (w/w) | NP embedding | Fe2O3] |
| PMMA cement | 10% | NP in silica beads | 21.6 |
| PMMA cement | 20% | NP in silica beads | 26.7 |
| Alginate hydrogel | 10% | NP in silica beads | 25.3 |
| Vinyl polymer (EVAL) | 5% | NP in silica beads | 20.9 |
| Vinyl polymer (EVAL) | 10% | NP in silica beads | 12.3 |
| Vinyl polymer (EVAL) | 20% | NP in silica beads | 11.2 |
| Vinyl polymer (EVAL) | 30% | NP in silica beads | 10.3 |
| Polyurethane | 10% | NP in silica beads | 37.4 |
| polymer | | | |
| PMMA cement | 20% | NP alone | 2.6 |
| Alginate hydrogel | 5% | NP alone | 6.4 |
| Vinyl polymer (EVAL) | 10% | NP alone | 2.3 |

### Example 13

### In Vivo Preliminary Experiment

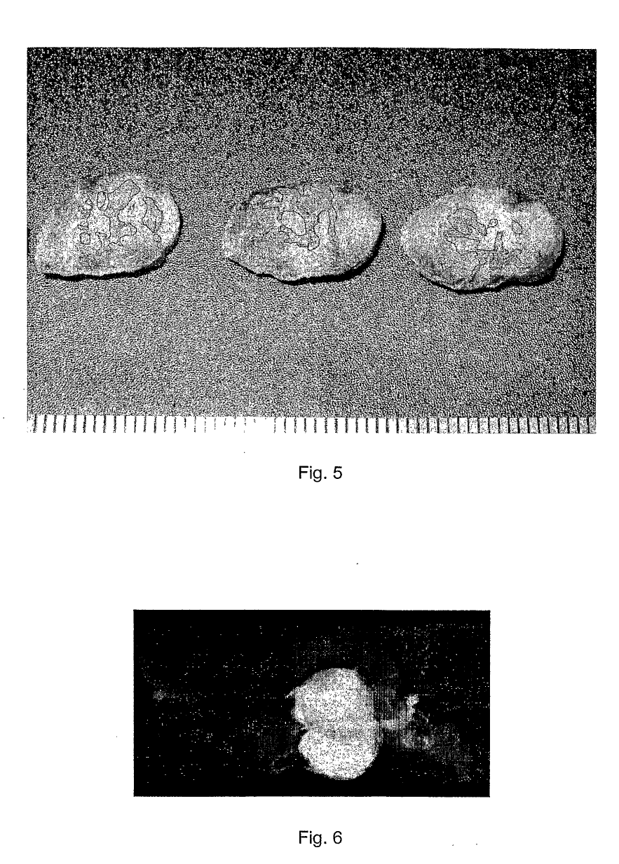

The formulation of EXAMPLE 3, containing 10% of iron oxide nanoparticles embedded in a silica matrix (beads), was injected into a mouse subcutaneous colon xenograft tumor T380. The ratio of the injected volume over the tumor volume was 40%. FIG. 5 shows the intratumoral distribution of the hyperthermic implants, as shown by the outlined areas. As expected, the liquid actually fills in the tumoral spaces before solidifying.

### Example 14

### Ex Vivo Experiment: Dog Prostate Model

Prostate cancer being a potential target for hyperthermic implant, an excised dog prostate was embolized with a 5% solution of polyurethane (Tecothane 75, Thermedics, USA) in N-methylpyrrolidone, containing 10% tantalum powder and 10% of iron oxide nanoparticles embedded in a silica matrix (beads). Direct puncture lead to a complete prostate filling as shown on the fluoroscopic image of FIG. 6.

### Example 15

### Drug Release from an Implant

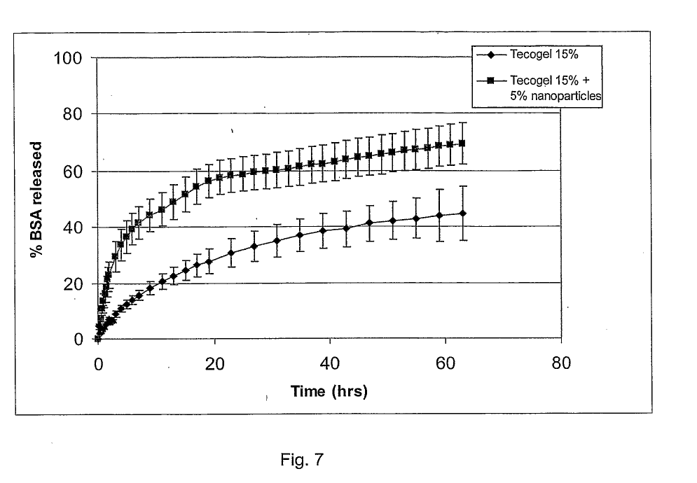

We prepared a solution of Tecogel (Thermedics, USA) 15% w/w in N-methyl pyrrolidone added with 10% w/w of iron oxide nanoparticles embedded in a silica matrix (beads) and 10% w/w bovine serum albumin (BSA) as a model drug. The solution was precipitated in a phosphate buffer. The BSA release was measured by spectroscopy at 270 nm. 80% of the BSA was released over 17 hrs as shown in FIG. 7. The release of BSA and smaller molecules such as antibiotics could also be prolonged using lower drug concentrations.

1.-22. (canceled)

23. An injectable formulation for treatment by hyperthermia comprising
a liquid carrier which is based on anyone of a precipitating polymer solution in water-miscible solvent, an in-situ polymerizing or crosslinking compound, a thermosetting compound and an hydrogel, and
heat-generating superparamagnetic iron oxide nanoparticles having a mean diameter not greater than 20 nm,
said injectable formulation being able to form in-situ an hyperthermic solid or semi-solid implant upon contact with a body fluid or tissue.

24. The injectable formulation according to claim 23, wherein the heat-generating superparamagnetic iron oxide nanoparticles have a mean diameter ranging from 5 to 15 nm.

25. The injectable formulation according to claim 24, wherein the heat-generating superparamagnetic iron oxide nanoparticles have a span of 1 or less, said span being defined as span=d90%−d10%/d50%, wherein d90%. d10% and D50% are the nanoparticle sizes in diameters, and the given percentage value is the percentage of particles smaller than that size.

26. The injectable formulation according to claim 23, wherein the heat-generating superparamagnetic iron oxide nanoparticles are maghemite nanoparticles, magnetite nanoparticles or a mixture thereof.

27. The injectable formulation according to claim 23, wherein the heat-generating superparamagnetic iron oxide nanoparticles have a non-spherical shape.

28. The injectable formulation according to claim 27, wherein the heat-generating superparamagnetic iron oxide nanoparticles have a diameter ratio of the larger diameter to the smaller diameter ranging from 1 to 3.

29. The injectable formulation according to claim 23, wherein the heat-generating superparamagnetic iron oxide nanoparticles are coated with a biocompatible polymer.

30. The injectable formulation according to claim 23, wherein the heat-generating superparamagnetic iron oxide nanoparticles are immobilized in organic or inorganic beads.

31. The injectable formulation according to claim 30, wherein the heat-generating superparamagnetic iron oxide nanoparticles are immobilized in silica beads.

32. The injectable formulation according to claim 31, wherein the silica beads immobilizing the heat-generating superparamagnetic iron oxide nanoparticles have a mean diameter ranging from 20 nm to 1 μm.

33. The injectable formulation according to claim 32, wherein the silica beads immobilizing the heat-generating superparamagnetic iron oxide nanoparticles have a mean diameter ranging from 300 nm to 800 nm.

34. The injectable formulation according to claim 31, wherein the iron oxide nanoparticles-containing silica beads are further coated with a biocompatible polymer.

35. The injectable formulation according to claim 23, wherein the liquid carrier is based on a precipitating polymer solution in water-miscible solvent consisting in a solution of a preformed polymer in an organic solvent which is able to precipitate in the tissue following exchange of the solvent with surrounding physiological water, thus being able to produce a polymer cast filling the tissue.

36. The injectable formulation according to claim 23, which further comprises a radiopacifier.

37. The injectable formulation according to claim 23, wherein the liquid carrier is based on a radiopaque polymer.

38. The injectable formulation according to claim 23, which further comprises drugs or biopharmaceuticals.

39. Use of an injectable formulation as defined in claim 23, for forming in-situ an hyperthermic solid or semi-solid implant.

40. Use of an injectable formulation according to claim 39, for forming in-situ an hyperthermic solid or semi-solid implant for treating a tumor.

41. Use of an injectable formulation according to claim 39, for forming in-situ an hyperthermic solid or semi-solid implant for treating a degenerative disc disease.

42. An hyperthermic solid or semi-solid implant, said implant being formed in-situ upon contact of the injectable formulation as defined in claim 23 with a body fluid or tissue, when said injectable formulation is injected into a body.

43. Use of an hyperthermic solid or semi-solid implant according to claim 42 for the treatment of a tumor.

44. Use of an hyperthermic solid or semi-solid implant according to claim 42 for the treatment of a degenerative disc.

45. A method for treating a tumor, which comprises forming in-situ a hyperthermic solid or semi-solid implant according to claim 42, and subjecting the hyperthermic solid or semi-solid implant to a heating procedure.

46. A process for preparing iron oxide nanoparticles-containing silica beads for use in the injectable formulation according to claim 31, said process comprising the steps of:
flocculating iron oxide nanoparticles in the presence of a controlled amount of poly(vinyl alcohol)(PVA) in order to give aggregates of iron oxide nanoparticles, reacting said aggregates of iron oxide nanoparticles with a silica precursor in order to give iron oxide nanoparticles-containing silica beads.

47. The injectable formulation according to claim 35, wherein the liquid carrier is based on a radiopaque polymer.
